(19) United States
(12) Patent Application Publication
(10) Pub. No.: US 2010/0055158 A1
(43) Pub. Date: Mar. 4, 2010

(54) Environmentally Activated Compositions, Articles and Methods

(75) Inventors: Ronald F. Vitaris, Worcester, MA (US); Harish A. Patel, Norfolk, MA (US); E. David Fink, Franklin, MA (US); Sharon A. Mulligan, Bristol, RI (US); Brian Dowd, Dedham, MA (US); Scott Orr, Franklin, MA (US); Chirag B. Shah, North Attleboro, MA (US)

Correspondence Address: TYCO HEALTHCARE GROUP LP, 15 HAMPSHIRE STREET, MANSFIELD, MA, 02048, US

(73) Assignee: Tyco Healthcare Group LP, Mansfield, MA (US)

(21) Appl. No.: 12/550,045

(22) Filed: Aug. 28, 2009

Related U.S. Application Data
(60) Provisional application No. 61/136,320, filed on Aug. 28, 2008.

(51) Int. Cl. A61K 9/70; A01N 33/12; A61K 31/155; A61K 38/02; A61P 17/02; A61P 31/04; A61P 31/02

(52) U.S. Cl. 424/447; 514/642; 514/635; 514/2

(57) ABSTRACT

An article includes a film layer; at least one layer of adhesive on at least one side of the film layer; a patch or strip comprising at least one antimicrobial agent, the patch or strip disposed on the same side of the film layer as the at least one layer of adhesive; and a relatively non-flexible sheet releasably attached to the side of the film layer opposite to the patch or strip. Alternatively, an article includes a film layer; a collagen layer; and a biodegradable hydrogel layer comprising PHMB; wherein the biodegradable hydrogel layer comprising PHMB is disposed between the film layer and the collagen layer

### FIELD

The present invention is directed to antimicrobial compositions, articles and methods.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

A variety of antimicrobial compositions, articles and methods have been suggested. However, such wound compositions and methods possess various deficiencies and shortcomings.

Thin film dressings are used for a wide range of applications. In their various configurations they have been used for skin tears, IV or catheter securement dressing attachment, and on surgical sites. However, many conventional thin film dressings do nothing to prevent site infection. Some thin film dressings are making there way into the marketplace that claim to provide a deterrent to microbial proliferation and infection. Each approaches the issue differently. One commercially available product blends iodophor into the adhesive. Another product is in the form of an absorbent disc that is impregnated with chlorihexidine gluconate that would be held in place with a thin film dressing. Yet another product provides a dressing where ionic silver is blended into the adhesive and film.

However, a need still exists in the art for compositions, devices and methods which have increased effectiveness in reducing and/or preventing development of unwanted microbial organisms, are safe, and provide for improved efficiencies in wound care management.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

### DEFINITIONS

As used herein, unless otherwise indicated, the terms “microbial organism” or “microbial” will be used to refer to microscopic organisms of matter, including fungal, bacterial and/or viral organisms. Thus, the term “antimicrobial” as used herein refers to a composition or agent that kills or otherwise inhibits the growth of such fungal, bacterial and/or viral organisms.

### SUMMARY

The present invention may address one or more of the problems and deficiencies of the prior art discussed above. However, it is contemplated that the invention may prove useful in addressing other problems and deficiencies, or provide benefits and advantages, in a number of technical areas. Therefore the claimed invention should not necessarily be construed as limited to addressing any of the particular problems or deficiencies discussed herein.

The present invention may optionally possess one or more of the following benefits or advantages: (i) compositions, laminates and methods which exhibit enhanced antimicrobial properties; (ii) compositions, laminates and methods which are effective and safe for human administration, as well as being readily available; and (iii) compositions, laminates and methods which enable more efficient wound care management.

The present invention may also optionally possess one or more of the following features, benefits and/or advantages.

A composition or wound dressing that targets antimicrobial activity to a specific area versus the entire dressing area.

Programmable release of antimicrobial agent(s). When multiple layers are used, a profile having an initial burst followed by sustained release would be beneficial to bring down the bacterial load initially and then provide continuous protection.

A gel forming action would control line leakage and provide painless removal in the area of insertion of an IV or catheter.

A dressing constructed so as to permit single-handed delivery.

A composition, article or method employing PHMB. PHMB is not harmful to normal skin flora and therefore does not disrupt epithilization. PHMB is cost effective enough to use as an everyday prophylactic. PHMB has no known resistance in over 60 years of common use; this allows it to be used on every wound with no major risk of resistance.

A transparent dressing so the clinician would be able to see the wound/exit site. Other products on the market visually occlude the site and require a dressing change for full site observation. The transparent nature of the strip and dressing would reduce dressing changes as the site/wound could be easily observed.

A dressing that includes a film that will dissolve based on moisture in the wound. The film can be gas permeable allowing the wound to breath during the healing process. The film may be formulated so that when moistened it will adhere to the skin and wound without the need for additional adhesive, making changing the dressing less traumatic to the patient's skin.

Controlling microbial levels at the wound site for optimizing the effects of a biodegradable collagen-containing dressing.

According to one aspect, the present invention provides an article comprising: a film layer; at least one layer of adhesive on at least one side of the film layer; a patch or strip comprising at least one antimicrobial agent, the patch or strip disposed on the same side of the film layer as the at least one layer of adhesive; and a relatively non-flexible sheet releasably attached to the side of the film layer opposite to the patch or strip.

According to an additional aspect, the present invention provides an article comprising: a film layer; a collagen layer; and a biodegradable hydrogel layer comprising PHMB; wherein the biodegradable hydrogel layer comprising PHMB is disposed between the film layer and the collagen layer.

According to additional aspects, the present invention provides wound dressings formed from any of the above-mentioned articles.

### BRIEF DESCRIPTION OF THE DRAWINGS

FIG. 1 is a schematic cross-sectional view of a laminate/dressing formed according to the principles of the present invention.

FIG. 2 is a schematic cross-sectional view of one embodiment of a component of the laminate/dressing of the present invention.

FIG. 3 is a schematic cross-sectional view of an alternative embodiment of a laminate/dressing of the present invention.

FIG. 4 is a top view of an alternative embodiment of the laminate/dressing of FIG. 3.

FIG. 5 is a top view of a laminate/dressing formed according to a further embodiment of the present invention.

FIG. 6 is a schematic cross-sectional view of a laminate/dressing formed according to an additional embodiment of the present invention.

FIG. 7 is a schematic cross-sectional view of the laminate/dressing formed according to yet another embodiment of the present invention.

FIG. 8 is a schematic cross-sectional view of a modified version, or alternative embodiment, of the laminate/dressing of FIG. 7.

FIG. 9 is a schematic cross-sectional view of still another embodiment of a laminate/dressing formed according to the present invention.

### DETAILED DESCRIPTION

According to certain embodiments, the present invention may be provided in the form of compositions, methods, or a novel thin film laminate or wound dressing that provides targeted release of an antimicrobial agent.

Any suitable film material may be utilized. The film could be made from any number of hydrophilic or hydrophobic materials, or polymers that would otherwise provide sufficient conformability and moisture vapor transmission rate (MVTR) suitable for the intended end use. Examples of possible polymers that could be useful are: polyester urethanes, polyether urethanes, polyolefins, EVA, PVC and metallocene. The film and/or entire laminate or dressing can be substantially transparent, at least to the extent so as to allow for the visual inspection of the skin surface lying underneath. The film and/or dressing can have any suitable geometry and/or dimensions. For example, the thin film dressing can comprise a thin (0.0005 inches to 0.0015 inches) flexible film.

Similarly, any suitable antimicrobial agent(s) may be utilized. For example, polymeric biguanides such as PHMB, PEHMB, or derivatives thereof can be utilized as the antimicrobial agent(s). Alternatively, certain metals, or compounds including such metals, such as silver, gold, copper or zinc may be used as the antimicrobial agent(s). It is additionally contemplated that the antimicrobial treatment could be a combination of a number of agents such as silver, PHMB, CHG, EDTA or other suitable antimicrobials such that a synergistic efficacy is realized.

According to certain embodiments, the antimicrobial agent(s) can comprise a cationic surfactant or a cationic quaternary ammonium compound. Non-limiting examples of such compounds include: benzalkonium chloride; benzethonium chloride; cetrimide; cetylpyridinium chloride; chlorphenoctium amsonate; dequalinium acetate; dequalinimum chloride; domiphen bromide; laurolinium acetate; methylbezethonim chloride; myristyl-gamma-picolinium chloride; ortaphnum chloride; triclobisonum chloride; cetalkonium chloride; dofanium chloride; tetraethylammonum bromide; didecyldimethylammonium chloride; tetraethylammonium bromide; dimethyldiallyl ammonium chloride; p-trialkylamioethyl styrene monomer; and trialkyl(p-vinylbenzyl) ammonium chloride.

According to further embodiments, the antimicrobial agent(s) can comprise a cationic surfactant or a polymeric quaternary ammonium compound. Non-limiting examples of such compounds include: poly(diallyl dimethyl ammonium chloride); poly(3-chloro-2 hydroxypropyl)methacryloxyethyl dimethyl-ammonium chloride; poly(acrylamide-methacryloxyethyl trimethyl-ammonium bromide; poly(butyl acrylate-methacryloxyethyl trimethylammonium bromide; poly(1-methyl-4-vinyl pyridinium bromide); poly(1-methyl-2-vinylpyridinium bromide); and poly(methylacryloxyethyl triethyl ammonium bromide).

According to additional alternative embodiments, the antimicrobial agent(s) can comprise a polyquaternium. Polyquaternium is a neologism used to emphasize the presence of quaternary ammonium centers in the polymer. Polyquaterniums are positively charged, and some have antimicrobial properties. There are currently at least 37 different known polymers under the polyquaternium designation. New polyquanterniums are identified periodically. Different polymers are distinguished by the numerical value that follows the word “polyquaternium.” Thus, the present invention contemplates the possible use of any of the currently known polyquaternium-1 through polyquaternium-37 substances, as well as future polyquanterniums, currently undesignated, falling under the broad definition or categorization noted above.

According to further embodiments, the antimicrobial agent(s) can comprise a cationic antimicrobial peptide, such as e-poly-I-lysine, magainin, cecropins, dermaseptin, pexiganan, iseganan, Oniganan, and defensin.

According to additional alternatives, the antimicrobial agent(s) can comprise amphoteric surfactants, such as include alkyl betaines, dodecyl betaine cocoampho glycinate, and cocamidopropyl betaine.

According to further embodiments, the antimicrobial agent(s) can comprise bromine based compounds such as poly(4-vinyl-N-alkyl pyridinium bromide); and poly(4-vinyl-N-hexylpyridinium bromide).

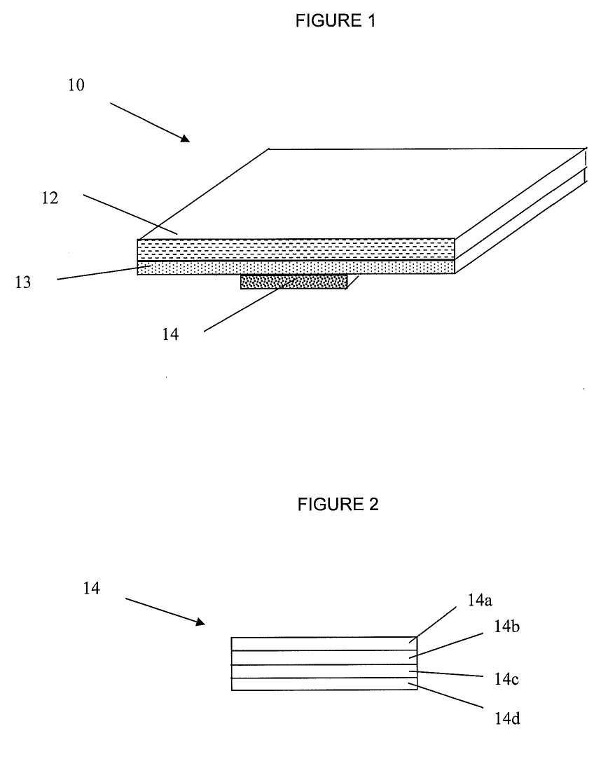

As illustrated in FIG. 1, according to further embodiments, the compositions, methods or laminate/dressing 10 may include, in addition to at least one aforementioned film layer 12, and at least one patch or strip 14 containing any suitable antimicrobial agent such as those described above.

The patch or strip 14 can be formed from any suitable material, or combination of materials. For example, the patch or strip may comprise woven and/or non-woven fibers. Suitable fibers may include natural fibers, synthetic fibers, or combinations thereof. Thus, suitable fibers can be formed from metal, ceramics, polymers, or natural materials. Non-limiting examples include: cotton, cellulose, polyester, polyethylene, polypropylene, PTFE, nylon, aramids, Kevlar, chitosan, alginates, poly(ethylene terephthate) (PET), acrylics, fluorocarbons, modacrylics, polyesters, rubber, saran, spandex, vinal, vinyon, rayon, acetate, triacetate, protein, flax, hemp, jute, ramie, manila, kapok, wool, or silk.

The fiber can have any suitable size, such as an effective diameter from 5 nm to 5 mm and the specific surface area can vary from 0.001 to 1000 m²/g. The cross section of the fibers can be delta, circular, fibrillated, or 4DG™ (commercially available from Fiber Innovation Technology, Inc., Johnson City, Tenn.; see also, Heather L. Paul et al., “Comparison of Thermal Insulation Performance of Fibrous Materials for the Advanced Space Suit,” Journal of Biomechanical Engineering, Volume 125, October 2003, Pages 639-647; entire contents incorporated herein by reference); or any other suitable shape.

Fibers can be combined in any suitable fashion, such as woven, non-woven, knit, felt, or braided. The fibers can be continuous fiber or tow, cut staple fiber, wet laid/paper, meltblown, flash spun fibrillated tape, spunbond, needle punched, carded, composite structures, thermal bonded, chemical bonded, hydroentangled, airlaid, drylaid, highloft, ultrasonically bonded, stitchbonded, or powderbonded.

The patch or strip 14 could comprise a foam. This foam could be composed of polyurethane, olefin, PVC, polypropylene, polyethylene, EVA, ESI, or other polymers. The foam could be a bead gas formed foam or a foam formed by any other suitable process. The foam could be open or closed cell, with 5 to 200 pores per inch (ppi). A closed cell foam could be formed by thermal, caustic or other means of reticulation. The density of the foam could vary from 1 to 5 lb/ft³.

The patch or strip 14 could also comprise a polymeric film. This film could be composed of many synthetic, manmade or natural polymers. The film could be perforated or fibrillated.

The antimicrobial-containing patch or strip 14 may be activated based on environmental conditions. Activation includes the at least partial release of an antimicrobial agent. One activation mode is moisture. Moisture reactive film technologies are currently used in breath strips, personal products and pharmaceuticals. Other possible activation mechanisms include: quick dissolving or disintegrating excipients in the strip; pH; enzymes; macrophages; ionic strength; moisture; and temperature. Antimicrobial-containing patches or strips 14 can be formulated having various levels of sensitivity, thickness, and/or various concentrations of antimicrobial agent. Thus, the patches or strips 14 can be provided with different antimicrobial agent elution rates.

According to an alternative embodiment, the at least one film layer 12 can be provided with printing or other indicia (not shown) to identify the product, active ingredients, shelf life, anticipated active antimicrobial lifetime, etc.

According to additional alternative embodiments, the laminate/dressing 10 may be provided with at least one layer of adhesive 13, on at least one side thereof. The antimicrobial-containing patch or strip 14 can be strategically placed on the adhesive side of the thin film dressing 10. Exact placement is determined by the intended end use or application. The adhesive 13 and/or the patch or strip 14 can be applied by any of a number of techniques know to those skilled in the art. Any suitable adhesive material can be utilized. Depending on the application, acrylics, PIB rubber and silicone adhesives can be used. The adhesive 13 may optionally be covered by a protective releasable paper or plastic sheet (not shown).

According to further alternative embodiments, the least one patch or strip 14 could be replaced with, or complimented by, a silicone sheet coated with antimicrobial agent or a non-woven fabric or laminate film treated with antimicrobial agent in any known manner (not shown).

As illustrated in FIG. 2, the antimicrobial-containing patch or strip 14 can comprise multiple layers (14a, 14b, 14c, 14d), one or more of the layers can have different antimicrobial agent elution rates and/or different active ingredients and/or different antimicrobial agents. Nonlimiting examples of suitable active ingredients that are be beneficial to wound healing include analgesics, vitamins, growth factors, haemostatic agents, and antimicrobials, etc. One possible multi-layer combination could provide sequential release of active ingredients such as antimicrobial agents, analgesics growth factors, etc.

In addition to providing targeted antimicrobial release, the patch or strip 14 could also be formulated with absorbent gel forming materials such as, but not limited to, polyethylene oxide, alginates, carboxymethylcellulose (CMC), polyvinylpyrrolidone (PVP), and cross-linked acrylic acids. The patch or strip 14 also could be formulated with biomaterials that influence the wound healing process. Examples include crosslinked collagen and hyaluronic acid. The patch or strip 14 also can contain materials that once activated generate materials that are antimicrobial and/or influence the wound healing process. Example include nitric oxide generating compounds. It is also envisioned that the formulation of the strip could include chelating agents to enhance the performance of the antimicrobial agent. Suitable chelating agents include, but are not limited to, EDTA.

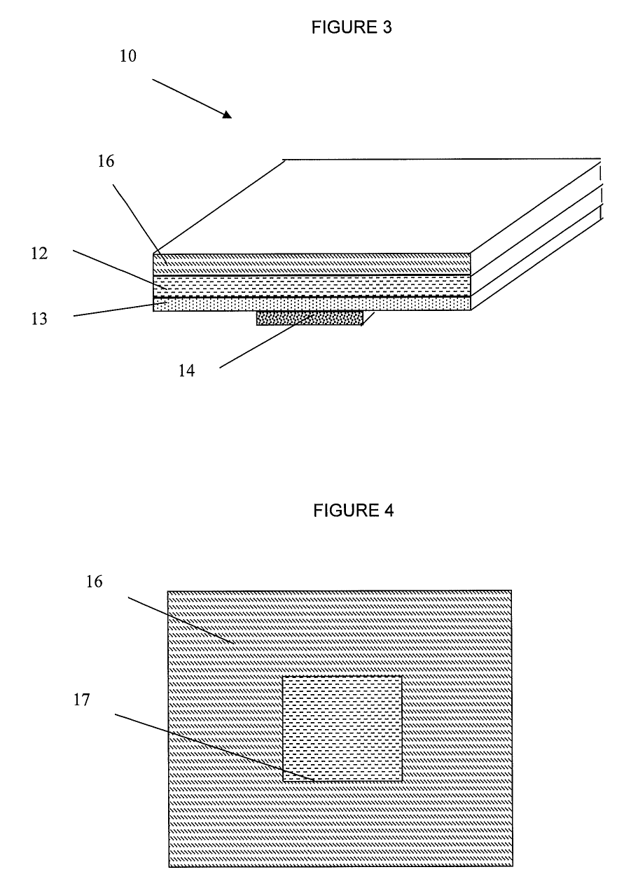

As illustrated for example in FIG. 3, according to further alternative embodiments, the laminate/dressing 10 can be provided with an optional sheet 16 of sufficient rigidity to promote “one handed” application of the completed laminate/dressing 10.

The sheet 16 can be formed of any suitable material having the desired degree of stiffness. For example, the sheet 16 can be formed from a polymer. One non-limiting example of a suitable polymer is ethylene vinyl acetate.

According to the illustrated embodiment, the sheet 16 is attached to a side of the film 12 which is opposite that of the adhesive 13. Any suitable technique can be utilized for attaching the sheet 16 to the film 12. One suitable technique is illustrated, for example, in U.S. Pat. No. 4,600,001, the entire contents of which are incorporated herein by reference. Thus, the sheet 16 can be substantially transparent and/or releasably attached to the film 12.

For non-catheter applications, a central area 17 of the sheet 16 can be removed to provide enhanced flexibility and improved clarity for seeing the wound through the laminate/dressing 10 (FIG. 4). For certain applications, such as P1CC lines, mid lines, central venous catheters, and dialysis catheters, the removed central section can be down-sized and located to one side of the dressing to allow for the incorporation of a fenestration into the dressing (not shown). Location and sizing of the viewing window and/or fenestration would vary per end use. Similarly, the laminate/dressing 10 could comprise a die cut section through all layers to facilitate insertion of a catheter or IV device.

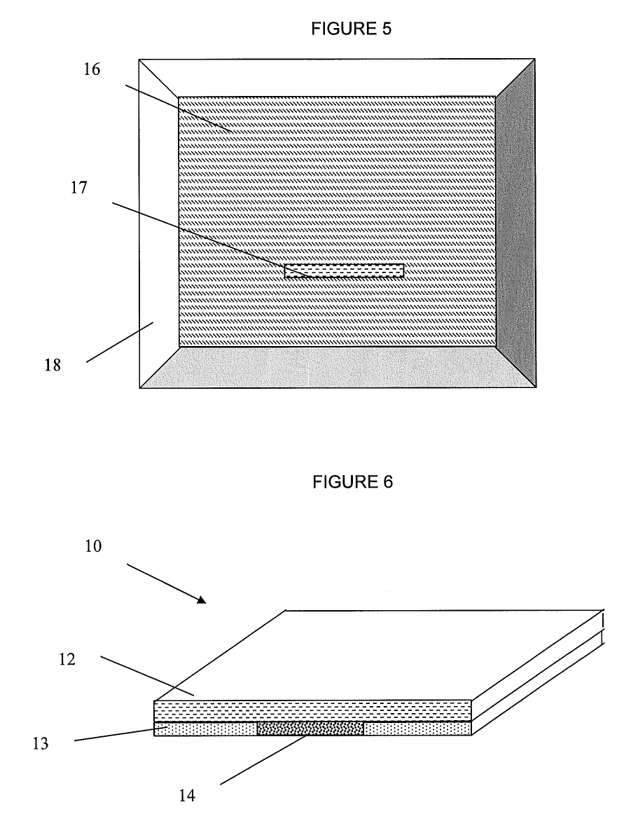

As illustrated for example in FIG. 5, the laminate/dressing 10 may further provide a foam, gel, or fabric type support pad 18 on the periphery of the dressing fastened above the edge of the film dressing and adhesive. The pad would prevent irritation of the skin from a catheter/IV device. Moreover, a strap or extra piece of adhesive could secure the catheter to the film rather than to the skin (not shown).

According to an additional embodiment of the present invention, the laminate/dressing 10 can be configured such that there is no adhesive 13 between the film 12 and the patch or strip 14. An example such embodiment is illustrated in FIG. 6. This construction would allow minimal trauma to the wound area in case of a dressing change.

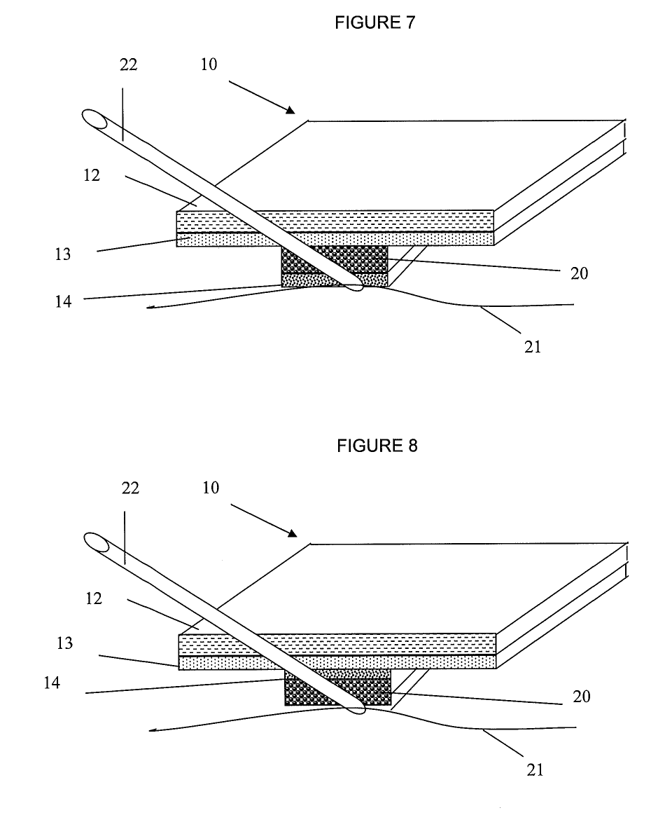

A laminate/dressing 10 formed according to the principles of the present invention may also include a piece of foam 20 or another absorbent material could be provided to add cushioning for different purposes, such as for catheter 22 exit and absorbency, as illustrated in FIG. 7. The patch or strip 14 could be placed either on the skin side 21 (FIG. 7) or the film 12 side (FIG. 8), wherein it would act as a barrier to microbes.

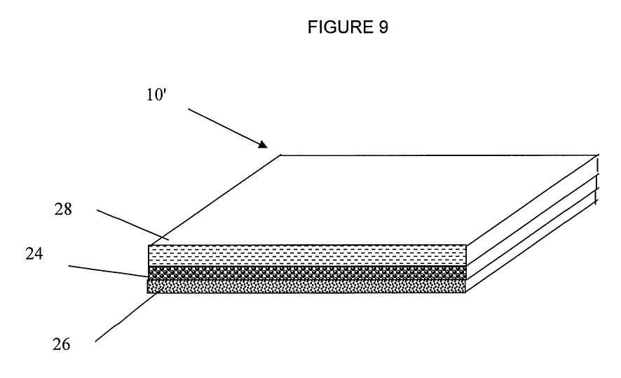

As noted above, the laminate/dressing may contain other wound healing agents in addition to one or more antimicrobial agents. For example, the laminate/dressing 10 may contain biodegradable collagen and PHMB to control microbial levels in it to prevent wound infection. According to one illustrative embodiment, as shown in FIG. 9, a laminate/dressing 10′ formed according to the principles of the present invention may comprise a collagen layer 24 sandwiched between a biodegradable hydrogel containing PHMB 26 to control microbial level in the wound, and outer film layer 28 to control external contamination. The outer film layer can be formed from any suitable material. For example, the film layer 28 can be formed from the same material(s) described above in connection with film layer 12.

Wound dressings can, of course, include additional active ingredients or agents such as, for example, a therapeutic agent, an organoleptic agent, a growth factor, an analgesic, a tissue scaffolding agent, a haemostatic agent, a protein inhibitor, collagen, enzymes, an anti-thrombogenic agent, an anesthetic, an anti-inflammatory agent, an anticancer agent, a vasodilation substance, a wound healing agent, an angiogenic agent, an angiostatic agent, an immune boosting agent, a skin sealing agent, an agent to induce directional bacterial growth, an agent to impart bactericidal or bacteriostatic activity, an electron transfer agent to destabilize or destroy the metabolic action of microbes and/or biofilm formation, combinations thereof and the like. Release of active agents may be triggered by a variety of means, such as, for example, an electric field or signal, temperature, time, pressure, moisture, light (e.g., ultra-violet light), ultrasound energy, sonication, combinations thereof and the like.

Any numbers expressing quantities of ingredients, constituents, reaction conditions, and so forth used in the specification are to be understood as being modified in all instances by the term “about”. Notwithstanding that the numerical ranges and parameters setting forth, the broad scope of the subject matter presented herein are approximations, the numerical values set forth are indicated as precisely as possible. Any numerical value, however, may inherently contain certain errors or inaccuracies as evident from the standard deviation found in their respective measurement techniques. None of the features recited herein should be interpreted as invoking 35 U.S.C. §112, ¶6, unless the term “means” is explicitly used.

Although the present invention has been described in connection with preferred embodiments thereof, it will be appreciated by those skilled in the art that additions, deletions, modifications, and substitutions not specifically described may be made without departing from the spirit and scope of the invention.

We claim:

1. An article comprising:
a film layer;
at least one layer of adhesive on at least one side of the film layer;
a patch or strip comprising at least one antimicrobial agent, the patch or strip disposed on the same side of the film layer as the at least one layer of adhesive; and
a relatively non-flexible sheet releasably attached to the side of the film layer opposite to the patch or strip.

2. The article of claim 1, wherein the relatively non-flexible sheet is substantially transparent.

3. The article of claim 3, wherein the non-flexible sheet comprises a window formed therein.

4. The article of claim 1, further comprising a support pad disposed on the periphery of the article.

5. The article of claim 1, further comprising at least one foam layer disposed on at least one side of the patch or strip.

6. The article of claim 5, wherein the foam layer is disposed between the patch or strip and the at least one layer of adhesive.

7. The article of claim 5, wherein the patch or strip is disposed between the foam layer and the at least one layer of adhesive.

8. The article of claim 1, wherein the film layer comprises: polyester urethane, polyether urethane, polyolefin, EVA, PVC or metallocene.

9. The article of claim 1, wherein the film layer has a thickness of about 0.0005 inches to about 0.0015 inches.

10. The article of claim 1, wherein the at least one antimicrobial agent comprises: a polymeric biguanide; a cationic quaternary ammonium compound, a polymeric quaternary ammonium compound; a polyquaternium; a cationic antimicrobial peptide; or combinations thereof.

11. The article of claim 1, wherein the patch or strip comprises: natural fibers, synthetic fibers, foam, natural polymers, synthetic polymers, or combinations thereof.

12. The article of claim 1, wherein the patch or strip is provided with an activation mechanism for release of the at least one antimicrobial agent, the activation mechanism comprising: moisture, dissolving or disintegrating excipients, pH, enzymes, macrophages, ionic strength, moisture, and temperature.

13. The article of claim 1, wherein the adhesive comprises: acrylic, PIB rubber or silicone.

14. The article of claim 1, wherein the patch or strip comprises a plurality of layers.

15. The article of claim 14, wherein the patch or strip is configured for sequential release of active ingredients.

16. The article of claim 1, wherein the patch or strip comprises polyethylene oxide, alginate, carboxymethylcellulose, polyvinylpyrrolidone, cross-linked acrylic acid, collagen or hyaluronic acid.

17. The article of claim 1, wherein the patch or strip further comprises a nitric oxide generating compound or a chelating agent.

18. The article of claim 1, wherein the relatively non-flexible sheet comprises ethylene vinyl acetate.

19. The article of claim 1, wherein there is no adhesive present between the patch or strip and the film layer.

20. A wound dressing comprising the article of claim 1.

21. An article comprising:
a film layer;
a collagen layer; and
a biodegradable hydrogel layer comprising PHMB;
wherein the biodegradable hydrogel layer comprising PHMB is disposed between the film layer and the collagen layer.

22. A wound dressing comprising the article of claim 21.
